(19) United States
(12) Patent Application Publication
(10) Pub. No.: US 2024/0207547 A1
(43) Pub. Date: Jun. 27, 2024

(54) SYSTEMS AND METHODS FOR MULTI-FREQUENCY OSCILLATORY VENTILATION

(71) Applicants: David KACZKA, Iowa City, IA (US); Jacob HERRMANN, Coralville, IA (US)

(72) Inventors: David KACZKA, Iowa City, IA (US); Jacob HERRMANN, Coralville, IA (US)

(21) Appl. No.: 18/598,784

(22) Filed: Mar. 7, 2024

Related U.S. Application Data
(63) Continuation of application No. 16/862,659, filed on Apr. 30, 2020, now Pat. No. 11,951,254.
Application No. 16/862,659 is a continuation of application No. 15/145,880, filed on May 4, 2016, now Pat. No. 10,675,423.
(60) Provisional application No. 62/163,737, filed on May 19, 2015.

(51) Int. Cl. A61M 16/00; A61M 16/10; A61M 16/20

(52) U.S. Cl. CPC A61M 16/0057; A61M 16/0006; A61M 16/0096; A61M 16/203; A61M 2016/0027; A61M 2016/0036; A61M 2016/102; A61M 2230/005; A61M 2230/202; A61M 2230/205; A61M 2230/208; A61M 2230/40; A61M 2230/46

(57) ABSTRACT

The present invention relates to systems and methods for multi-frequency oscillatory ventilation (MFOV). The system uses a broadband flow waveform more suitable for the heterogeneous mechanics of the lung. The system provides more efficient gas exchange and enhanced lung recruitment at lower airway pressures.

### CROSS-REFERENCE TO RELATED APPLICATIONS

This application is a continuation of U.S. patent application Ser. No. 16/862,659 filed Apr. 30, 2020 which is a continuation of U.S. patent application Ser. No. 15/145,880 filed May 4, 2016 which application claims priority to U.S. Provisional Application 62/163,737 filed on May 19, 2015 the contents of both are hereby incorporated by reference herein.

### GOVERNMENT LICENSE RIGHTS

This invention was made with government support under Grant No. UM1HL108724 awarded by the National Institutes of Health. The government has certain rights in the invention.

### COPYRIGHT STATEMENT

This patent disclosure contains material that is subject to copyright protection. The copyright owner has no objection to the facsimile reproduction by anyone of the patent document or the patent disclosure as it appears in the U.S. Patent and Trademark Office patent file or records, but otherwise reserves any and all copyright rights.

### FIELD OF THE INVENTION

This disclosure relates generally to ventilators for supplying gas to facilitate and support respiration, and particularly to ventilators which employ multi-frequency ventilation with specifically tuned frequencies and amplitude and phase control over the applied waveform.

### BACKGROUND

Respiratory failure from acute respiratory distress syndrome (ARDS) is associated with high mortality acutely (up to 40%), and accounts for about 4 million ICU days annually in the U.S. ARDS survivors have substantial morbidity, and may have long-term physical and mental health impairments. ARDS imposes significant burdens on public health resources worldwide, and only minimal improvements in outcomes have occurred over recent decades.

Risks for developing ARDS include a diverse range of predisposing factors and initiating insults, such aspiration, pneumonia, trauma, sepsis, pancreatitis, inhalation injury, transfusion, and burns. Regardless of etiology, ARDS results in progressive deterioration in lung function towards a final common pathway: respiratory failure characterized by alveolar flooding, derecruitment, reduced compliance, increased shunting and dead space, and life-threatening hypoxemia. A key pathologic feature of ARDS is the heterogeneity of local injury severity and regional mechanical properties.

The mainstay of treatment for ARDS is endotracheal intubation and conventional mechanical ventilation (CMV). However CMV may exacerbate existing lung injury, due to cyclic, intratidal overdistention and repeated, asynchronous opening and closing of airways with each inflation. The mechanical stresses associated with these phenomena result in the release of cytokines and other inflammatory mediators that may exacerbate lung injury. This ventilator-associated lung injury (VALI) is thus a direct result of the mechanical heterogeneity of injured parenchyma, leading to maldistribution of ventilation and corresponding impairments in gas exchange.

Ventilation strategies that limit this end-expiratory derecruitment and end-inspiratory overdistension are the only interventions to have significantly reduced the morbidity and mortality of ARDS, using appropriate levels of positive end expiratory pressure (PEEP) to limit end-expiratory opening and closing and low tidal volumes (VT's) to reduce inspiratory overdistention. Such ‘protective’ ventilation strategies, however, may result in significant hypoventilation of the injured lung, due to increased deadspace and ventilation to perfusion ratio (\dot{V}/\dot{Q}) abnormalities. Most lung protective strategies for ventilator management use algorithmic, ‘one size fits all’ approaches, based on height, weight, or global arterial oxygenation. Adjustments to VT or PEEP based on such criteria provide little insight into how such interventions impact regional gas transport in the injured lung, or how to customize a ventilator management strategy for an individual patient's pathophysiology. For example, the optimal level of PEEP depends much more on the unique pattern of injury and amount of recruitable lung, rather than on oxygenation alone.

There is a continuing need for improvement in ventilation techniques to treat a variety of lung conditions and injuries.

### SUMMARY

The present invention relates to systems and methods for improving lung function and gas exchange. Volume oscillations are applied at multiple frequencies substantially simultaneously, rather than at a single high frequency, to provide more even distribution of ventilation to different lung regions in accordance with local mechanical properties. Ventilating an injured lung with a broadband waveform, for example, optimizes gas transport to the periphery, and thereby improves oxygenation and \dot{V}/\dot{Q} matching. ‘Multi-Frequency Oscillatory Ventilation’ (MFOV) is specifically configured to complement the heterogeneous mechanics of the injured lung. In effect, MFOV allows the local impedances of the injured parenchyma to selectively filter out flows of ‘less-desirable’ frequencies, and allows flows at frequencies more effective for a particular region to participate in gas exchange. With further adjustments in oscillatory pressure amplitude and mean airway pressure, MFOV improves gas exchange in the injured lung while minimizing the detrimental effects of cyclic alveolar overdistention and derecruitment.

In one implementation, a system is described with an oscillatory ventilator configured for oscillating at plurality of specifically tuned frequencies simultaneously a ventilation gas for delivery to a pulmonary region of a patient. A ventilator control system, in communication with the oscillatory ventilator, controls a waveform input for the oscillatory ventilator. The waveform input comprises the plurality of specifically tuned frequencies, and an amplitude and a phase associated with each frequency that is alterable in response to physical or physiologic changes in the patient. A sensor can be provided for measuring the ventilation gas being delivered to a pulmonary region of the patient and providing the measurement to the ventilator control system for use in producing the waveform.

### BRIEF DESCRIPTION OF THE DRAWINGS

FIG. 1A shows an MFOV system in accordance with preferred embodiments of the invention;

FIG. 1B schematically illustrates a system for controlling delivery of multi-frequency therapeutic ventilation to a patient;

FIG. 2 schematically illustrates selective delivery of different frequency components to different regions of the lung in accordance with preferred embodiments of the invention;

FIG. 3 illustrates a method of treating a patient using a hybrid MFOV system in accordance with various embodiments;

FIG. 4 schematically illustrates amplitude and phase control of MFOV waveforms;

FIG. 5 illustrates the use of different ventilator frequency modes including conventional, high-frequency, and multi-frequency modes;

FIG. 6 illustrates acinar flow as a function of frequency and tissue stiffness;

FIGS. 7A and 7B illustrate a computed tomography image and a 3D model of a lung including peripheral airways;

FIG. 8 illustrates CT images of lung expansion and contraction before and after injury;

FIG. 9 illustrates anatomic distributions of acinar O2 and CO2 partial pressures of a lung in accordance with preferred embodiments of the invention;

FIGS. 10A-10C graphically illustrate root mean square of delivered volume, ventilatory cost function, and mean airway pressure, respectively;

FIGS. 11A-11D show representative examples of time-domain tracings (left panels A and C) and magnitude spectra (right panels B and D) for SFOV and MFOV volume waveforms, as generated by an oscillator in accordance with the invention;

FIG. 12 illustrates a method for adjusting mean airway pressure (P̅ₐₒ) and fraction of inspired oxygen concentration (FiO₂) based on arterial oxygen saturation (SpO₂);

FIGS. 13A-13I illustrate (A) arterial pH, (B) arterial CO₂ tension (PaCO₂) (C) arterial O₂ tension (PₐO₂), (D) peak-to-peak tidal volume normalized by weight, (VppWt−1), (E) root mean square volume normalized by weight (VmsWt−1), (F) ventilatory cost function (Vc), (G) mean airway pressure (P̅ₐₒ), (H) oxygenation index (OI), and (I) dynamic respiratory system elastance (Edyn) versus time during single-frequency oscillatory ventilation (SFOV, closed symbols) and multi-frequency oscillatory ventilation (MFOV, open symbols). An asterisk (*) indicates significant difference between SFOV and MFOV modalities, using two-way repeated measures ANOVA with Tukey HSD criterion. All data are expressed as mean+S.E.

FIG. 14 shows fractional distribution of ventilation within 32 slices of the left (gray bars) and right (white bars) hemithoraces following one hour of SFOV and MFOV modalities (i.e., at 90 and 180 minutes of the protocol) for Groups A (n=6) and B (n=6). Direction from dependent to nondependent slices is indicated by vertical arrow; Data are expressed as mean+S.D. of functional EIT values; Anatomic directions are denoted as R: right; L: left; V; ventral; D: dorsal.

FIGS. 15A-15B illustrate geometric centers of ventilation within the fEIT images (32×32 pixels) following one hour of SFOV and MFOV modalities (i.e., at 90 and 180 minutes of the protocol) for Groups A (n=6) and B (n=6); Pixels are referenced relative to the upper left original for each panel; Anatomic directions are denoted as R: right; L: left; V: ventral; D: dorsal; Data are expressed as mean+S.D. of the geometric centers of ventilation

### DETAIL DESCRIPTION

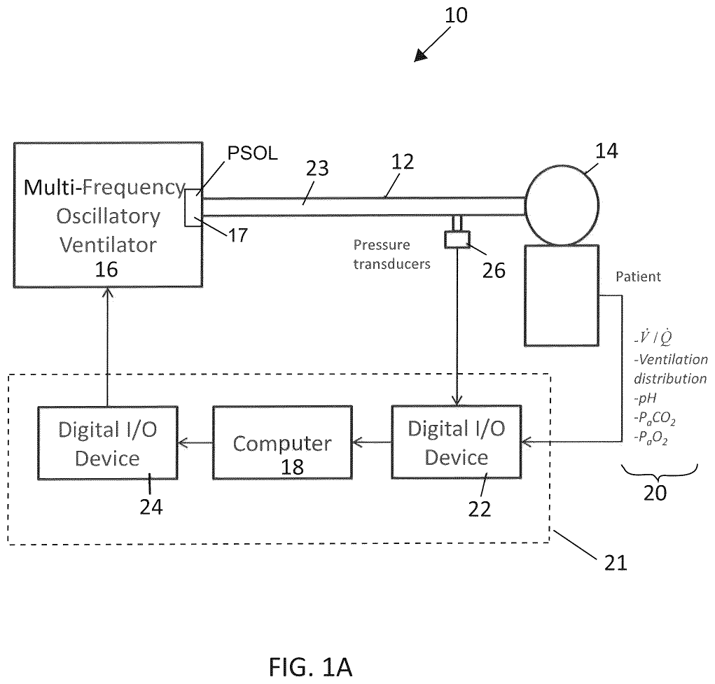

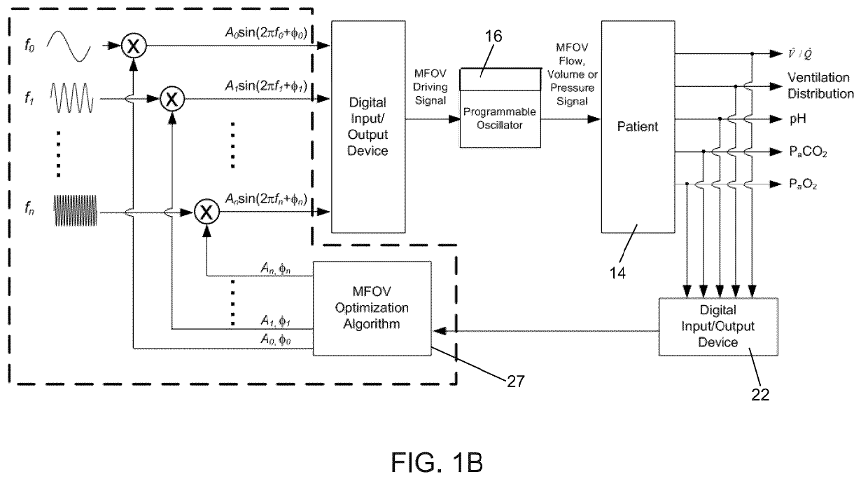

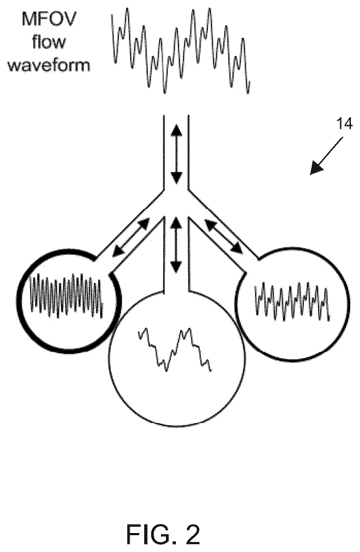

The present invention relates to combinations of oscillatory frequencies, amplitudes, and phases by which multi-frequency oscillatory ventilation (MFOV) can support gas exchange. MFOV relates to ventilation frequency for gas exchange that varies from region-to-region, depending on gravity, airway branching, and local mechanical properties of the tissue. Ventilator system 10 includes an oscillatory ventilator 16 to deliver ventilation gas 23 under pressure to the airway of a patient 14 as shown generally in FIG. 1A. The ventilator system 10 can include an oscillatory ventilator 16 operative to apply one or more specifically tuned frequencies to the gas delivered through tubing system 12 to the patient 14. Specifically tuned frequencies may be each determined and applied independent of each other, which means that they can each be fundamental frequencies, as opposed to a single fundamental frequency and multiple harmonic frequencies. Physiological characteristics 20 (e.g. Q, Ventilation Distribution, pH, PₐCO₂, and PₐO₂) can be measured on the patient 14 with corresponding sensors and operating characteristics (e.g. gas pressure) can measured by one or more transducers 26. The measurements can be delivered to input device 22 (if the measurements are in analog form, input device 22 can include an analog to digital convertor). The data from input device 22 is received by a computer 18 for recordal and selectively used with an output device 24 to control the operation of oscillatory ventilator 16. The computer 18 executes stored software/firmware to control the operating parameters of oscillatory ventilator 16, including the frequencies generated by the oscillatory ventilator 16, as shown schematically in FIG. 1B and FIG. 2. The software can include optimization software 27 to control the amplitude and phase characteristics of the applied waveform input (shown in FIG. 2). This can be done digitally, as shown, with digitally implemented signal generators for producing n-waveforms each with a frequency, phase, and amplitude. As the airways of each lung have unique heterogeneous features, the different frequencies applied during a therapeutic sequence can treat different regions simultaneously. The applied frequencies can be staggered or temporally offset by a time (t) or simultaneously applied to treat different conditions. In accordance with certain implementations, one or more therapeutic drugs may be entrained within the gas flow to enable direct treatment of affected organs, or properties of the gas flow including, but not limited to, moisture level may be altered to effect positive therapeutic outcomes in the patient.

Ventilator system 10 can be implemented with any type of ventilator or oscillator, and for low frequency ventilation or high frequency ventilation. The frequency levels for high frequency ventilation compared to low frequency ventilation depends on a number of factors including the species, the alveolar ventilation of the species, and the volume of the conducting airways. The transition from low frequency ventilation to high frequency ventilation is defined as: fₜ=5*(V′_{A}/V_{D}); where V′_{A} is the alveolar ventilation of the species, and V_{D} is the anatomic dead space volume (i.e., the volume of the conducting airways).

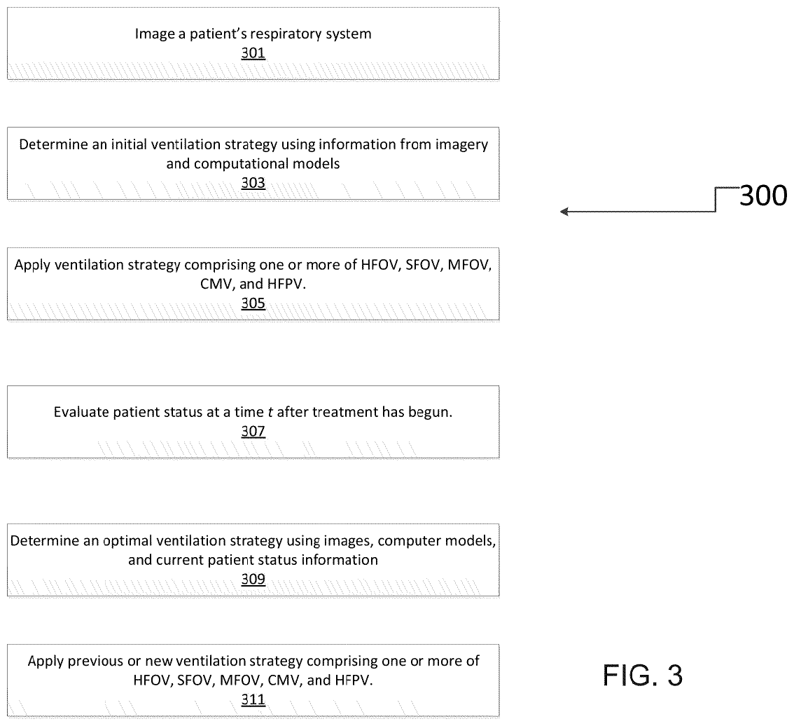

FIG. 3 illustrates a method 300 of treating lung injury or disease in a patient 14. Using, for example but not limited to, CT imaging or EIT, the practitioner may image 301 a patient's 14 respiratory system. With the information gathered from this imagery, the practitioner can determine 303 an initial ventilation strategy by combining the imagery with computational models. The models and related methods enable the practitioner to apply 305 a ventilation strategy comprising one or more of high-frequency oscillatory ventilation (HFOV), single-frequency oscillatory ventilation (SFOV), MFOV, conventional mechanical ventilation (CMV), and high-frequency percussive ventilation (HFPV). After the patient 14 has been treated for a suitable period of time, the practitioner may evaluate 307 the patient's status. Evaluation of the patient 14 may include additional imagery, visually assessment of the patient 14, or acquisition of patient 14 output values such as blood pH, HCO₃⁻, PₐCO₂, PₐO₂, and SpO₂. Based on the results of the evaluation, the practitioner may determine 309 an optimal ventilation strategy using images, computer models, and current patient status information. The optimal ventilation strategy may be the same or different from the initial ventilation strategy. The practitioner can choose to apply 311 the previous ventilation strategy or a new ventilation strategy comprising one or more of HFOV, SFOV, MFOV, CMV, and HFPV.

Mathematical models of biological systems are useful for defining complex relationships between variables and their effects on outcomes, predicting behavior over ranges of conditions, and helping design better experiments. MFOV assumes that the ideal ventilation frequency for gas exchange in the lung will vary from region-to-region, depending on gravity, airway branching, and local mechanical properties. The design of optimal MFOV waveforms in the injured lung thus requires an understanding of the relationship between heterogeneous lung injury and regional ventilation distribution. Advective and diffusive ventilation distribution to the parenchyma under varying degrees of heterogeneous lung injury are simulated using various computational models, incorporating specific three-dimensional information for individual airway and vascular segments. Nodes in the airway tree will account for the viscous dissipation and advective acceleration of gas flow in cylindrical airway conduits, as well as viscoelastic airway walls and parenchymal tissues. To assess regional ventilation to individual acini in the model, the entire tree is traversed using a recursive flow divider algorithm. An example of such an algorithm can be found in Amini, et al., “Intratidal Overdistention and Derecruitment in the Injured Lung: A Simulation Study,” Manuscript Submitted to IEEE on Dec. 4, 2015, the contents of which are hereby incorporated by reference herein. Corresponding spatial distributions of acinar pressures and flows are simulated throughout the lung for various MFOV waveforms. Since a portion of the MFOV waveform will be lost to gas compression and airway wall distention, the amount of flow available for ‘useful’ ventilation at the alveoli will be limited. Such flow losses will not be uniformly distributed across the lung, and will depend on the geometry of airway branching, frequency of oscillations, as well as gravity and local mechanical properties in this heterogeneously injured lung model. Nonlinear alterations in regional impedance are assumed to arise from cyclic variations in airway size, parenchymal strain-stiffening, and acinar recruitment/derecruitment. Acini are allowed to transition between recruited and derecruited states when exposed to stochastically-determined critical opening and closing pressures, respectively. The amplitude and phase components of various MFOV waveforms presented to the computational lung model are iteratively adjusted, using a nonlinear gradient search technique. Several robust optimization criteria are employed, including the distribution widths of net acinar ventilation, \dot{V}/\dot{Q} ratios, regional alveolar pressures and strains, as well as steady-state values for PₐO₂, PₐCO₂, and pH. Implementation of the algorithm in three dimensions allow for the identification of those anatomic regions that benefit the most from specific MFOV frequencies, as well as those regions which may experience overdistention or atelectrauma. These simulations are used to recommend and augment global strategies for MFOV management, based on the mechanics and recruitment patterns of injured lungs.

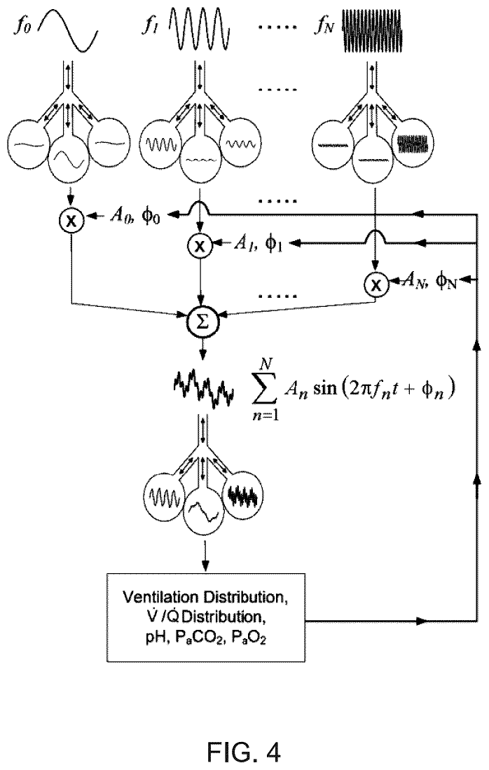

Turning to FIG. 4, each MFOV waveform input can be constructed using up to N sinusoids with different amplitude, frequency, and phase components (Aₙ, fₙ, and Φₙ, respectively). Each component that contributes to the overall MFOV waveform input may be substantially constant over the treatment regimen or may change with time, and the composition of the MFOV waveform input itself (including the number of sinusoids) may change over time. These changes may be prescribed in advance and specifically tuned as part of a multi-step treatment program, or the changes may be made in response to visual observations of patient 14 status or measured physical indicators of patient 14 status including blood pH, HCO₃⁻, PₐCO₂, PₐO₂, and SₐO₂.

The multi-frequency method can be used with conventional ventilation (CMV), with single (high) frequency and multi-frequency periods which can be monitored by selected measurement techniques as described herein and as shown in FIG. 5. According to various embodiments, a treatment regimen may comprise alternating periods of CMV, SFOV, and/or MFOV. The SFOV and MFOV treatments in any given period may have substantially constant frequency, amplitude, and phase components or these components may change within or between periods. According to certain embodiments, a given period may comprise more than one technique simultaneously, i.e., CMV may be provided at the same time as SFOV or MFOV.

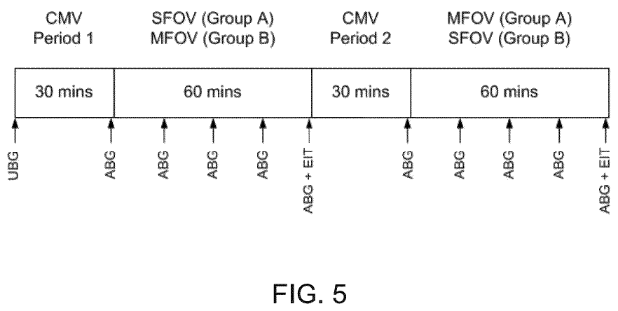

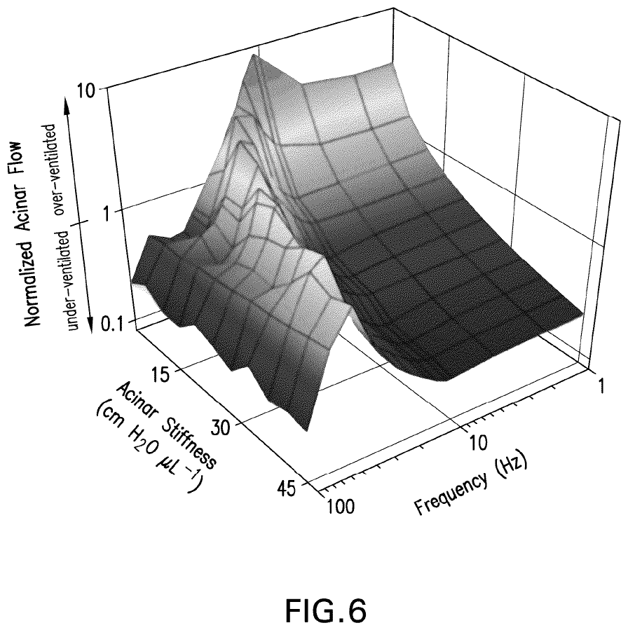

It is difficult to determine the influence of certain factors on the wide distribution of acinar flows and pressures in an injured lung. Mathematical models of biological systems are useful for defining complex relationships between variables and their effects on outcomes, and for predicting behavior over ranges of conditions. Thus, computational modeling can be used for MFOV waveforms in the injured lung, to simulate the complex relationship between mechanical heterogeneity and regional ventilation distribution. High-fidelity computational models of mammalian lungs are used for predicting gas transport and exchange during MFOV. Turning to FIG. 6, normalized acinar flow is illustrated as a function of both frequency and tissue stiffness. In accordance with various embodiments, the acinar flow can be modeled and simulated using an asymmetric, binary-tree data structure with terminal tissue elements corresponding to heterogeneously injured lung tissue. By determining acinar stiffness through computational modeling and imaging of the patient's 14 respiratory system, the model of normalized acinar flow illustrated in FIG. 6 can be used to determine the optimal frequency to strike a balance between under-inflation and over-ventilation. Additional details regarding modeling in mammalian systems is described in Amini et al., “Impact of Ventilation Frequency and Parenchymal Stiffness on Flow and Pressure Distribution in a Canine Lung Model”, Annals of Biomedical Engineering, Vol. 41, No. 12 (2013), pp. 2699-2711, the entire contents of which is incorporated herein by reference.

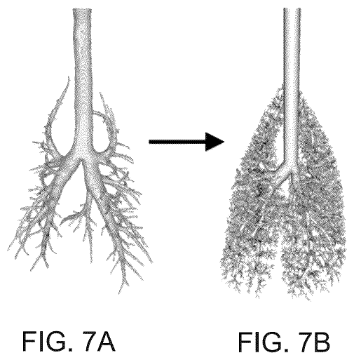

Computational lung models and a recursive flow divider method are combined to simulate advective and diffusive ventilation distribution to the parenchyma under varying degrees of heterogeneous lung injury. Three-dimensional information for individual airway and vascular segments using a database of whole-lung high resolution CT images in healthy and injured mammalian lungs, as well as newly acquired CT images can be utilized. An example of a segmented airway tree obtained from a CT image of a dog lung inflated to 35 cm H2O is shown in FIG. 7A. Details for methods of CT image utilization can be found in Black, et al., “Impact of Positive-End Expiratory Pressure During Heterogeneous Lung Injury: Insights from Computed Tomographic Image Functional Modeling,” Annals of Biomedical Engineering, Vol. 36, No. 6, June 2009, pp. 980-991, the entire contents of which is incorporated herein by reference. These images will allow us to develop an airway tree down to diameters of 3 mm. Smaller airways may be modeled using a CT image-based space-filling method. This method allows the use of a greater number of distal airways down to the level of the terminal bronchi within distinct lobular sections. An example of the use of the space-filling method to flesh out peripheral airways in a 3-D model reconstruction of a lung is shown in FIG. 7B.

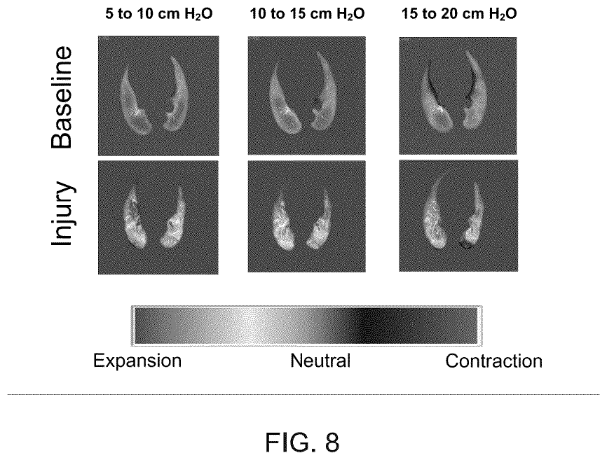

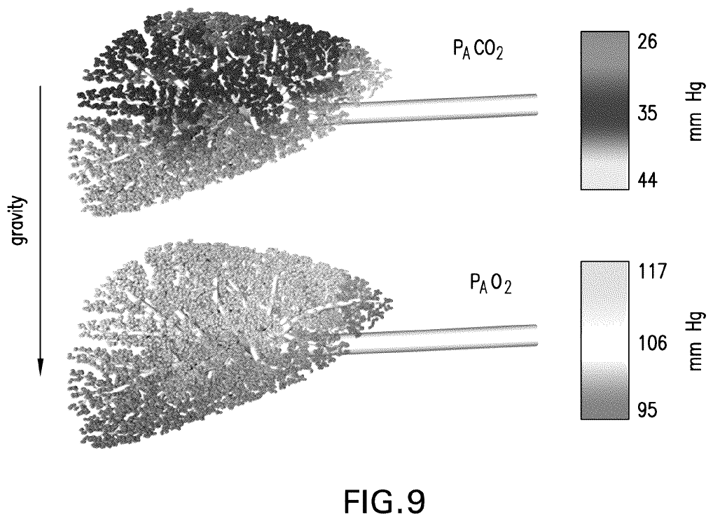

Shown in FIGS. 8 and 9 are methods used to model the therapeutic treatment to be delivered to an individual patient 14. FIG. 8 illustrates regional lung expansion based on CT image registration in a canine lung before and after lung injury for 3 different inflation pressure ranges. This registered comparison between pre- and post-injury conditions shows the variation in regional expansion/contraction. Such regional variations are the result of the heterogeneity of the lung injury and the lung itself and clearly illustrate why the multi-frequency approach can help by synthesizing treatment regimens directed to different lung morphologies. FIG. 9 illustrates a simulation of the anatomic distributions of acinar O₂ and CO₂ partial pressures for a canine lung model ventilated with a V_{T} of 10 cc kg⁻¹ at 20 breaths min⁻¹ using the gas exchange method. The granularity of the method (operating at the level of the acini) helps to better predict and target areas of different morphology using the MFOV technique.

To develop a mammalian model, thirteen preterm lambs (128 to 130 days gestation, term 150 days) weighing 3.15±0.39 kg were delivered via Cesarean section from anaesthetized ewes. Ewes had received 150 mg medroxyprogesterone intramuscularly at 100 days gestation, and 0.15 mg kg-1 betamethasone 72 and 48 hours prior to delivery. After delivery of the fetal head, the carotid and external jugular vessels were catheterized. Each lamb was intubated with a 4.5 mm cuffed endotracheal tube (ETT) via direct laryngoscopy. Lung liquid was manually aspirated using a 50 mL syringe. The fetal thorax was then exteriorized from the uterus and dried. In preparation for the EIT measurements, sixteen 23G needle electrodes were placed subcutaneously at equidistant locations around the chest ˜3 cm above the xiphisternum. The electrodes were secured using a 5-cm-wide self-adherent bandage (Coban; 3M, St. Paul, MN). After cutting the umbilical cord, the lamb was weighed, placed prone in a radiant warmer, and given two recruitment maneuvers of 30 cmH2O of 10 second duration. The lamb was then stabilized for 30 minutes on CMV at a rate of 50 min-1, VT of 7 mL kg-1, PEEP of 6 cmH2O, and FiO2 of 30% (Fabian, ACUTRONIC Medical Systems AG, Switzerland). General anesthesia and suppression of spontaneous breathing efforts was maintained using continuous intravenous infusions of propofol (1 to 2 mg kg-1 min-1) and remifentanil (0.5 to 1.0 μg kg-1 min-1). Heart rate, arterial blood pressure, rectal temperature, and pre-ductal oxygen saturation (SpO2, as measured at the right ear) were monitored continuously (HP48S; Hewlett Packard, Andover, MA). Pressure (Pao) and flow (\dot{V}) were sampled at 200 Hz by the ventilator using a hot wire anemometer (Florian, Acutronics, Herzel, Switzerland) and pressure port at the proximal end of the endotracheal tube. Examples of instruments to measure pressure and flow include, but are not limited to, pressure transducers such as the PXLA75DN or PXLA02X5DN (Honeywell), Codman (Johnson & Johnson, Raynham, MA), 33NA002D (IC Sensors, Milpitas, CA), or LCVR-0002 (Celesco, Canoga Park, CA). In addition, a range of mesh- or screen-type heated pneumotachographs (e.g., Hans-Rudolph 3700) may be used. Volume delivered at the airway opening (V) was determined using trapezoidal integration of the sampled V signal.

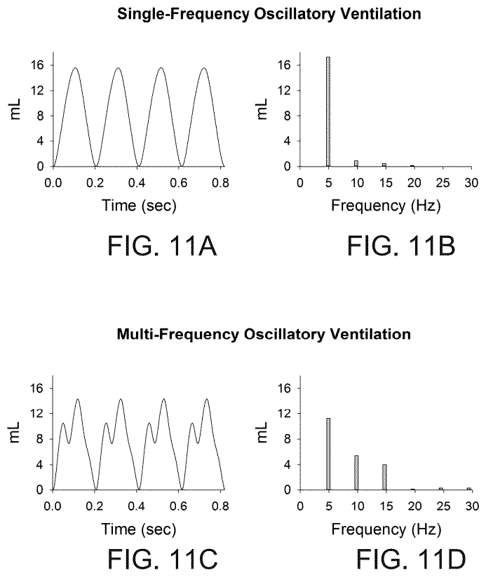

Following the initial 30 minute period of CMV, each lamb was randomized to receive either SFOV or MFOV for 60 minutes, followed by a 30 minute CMV washout period and crossover to the alternative regimen for 60 minutes (FIG. 5). The SFOV waveform can consist of a pure sinusoidal volume waveform at 5 Hz (applied using a commercial Fabian system, ACUTRONIC AG), while the MFOV waveform can include a 5 Hz fundamental with additional energy at 10 Hz and 15 Hz (FIGS. 11A-11D). The MFOV waveform may be generated by modifications of the Fabian firmware to apply the method described herein. In accordance with various embodiments, the CMV, SFOV, or MFOV waveforms may be generated by a variety of ventilators, oscillators, and flow interrupters including, but not limited to, the Humming V or Novalung (Metran, Saitama, Japan), Sensormedics (Sensormedics, Yorba Linda, CA), STEPHAN HF (F. Stephan Medizintechnik, Gackenbach, Germany), Infant Star (Infrasonics, San Diego, CA), Babylog (Draegerwerk, Lubeck, Germany), Dufour (Valleneuve d'Aseq, France), or any other device that is or may be modified to become suitable to produce ventilator waveforms of the type described herein. These devices may generate the air flow with a proportional solenoid valve or a piston solenoid. Peak-to-peak volume excursions (Vpp) were determined for both SFOV and MFOV as the difference between the maximum and minimum of the volume signal. After subtraction of the non-zero mean of the volume signal (V̅), the root mean square of the discretized volume (Vᵣₘₛ) was determined as:

$${V}_{rms} = \sqrt{\frac{1}{N} {\sum }_{n = 1}^{N} {( {V}_{n} - \overline{V} )}^{2}} \qquad ( 1 )$$

where Vₙ denotes the discretized volume waveform and N is the number of data points per each 0.2 second period of the SFOV or MFOV waveform. Dynamic respiratory system elastance (E_{dyn}) at 5 Hz was computed from 2 minute samples of Pₐₒ and \dot{V} waveforms at 15 minute intervals during SFOV and MFOV, using a periodogram technique with a 1-second rectangular window and 80% overlap.

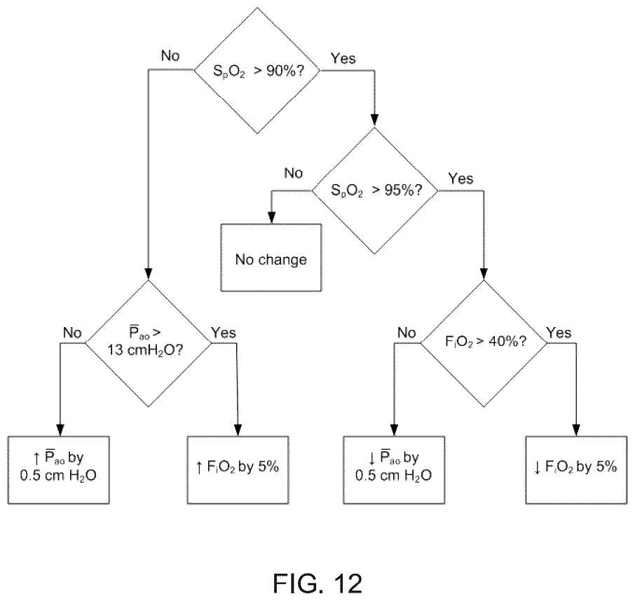

Arterial pH, carbon dioxide tension (PₐCO₂), and oxygen tension (PₐO₂) were obtained every 15 minutes throughout the entire protocol. During SFOV and MFOV, mean airway pressure (P̅ₐₒ) and % inspired O₂ fraction (FᵢO₂) were adjusted as needed according to the method shown in FIG. 12. The oxygenation index (OI) was calculated as:

$$O I = \frac{{F}_{i} {O}_{2} {\overline{P}}_{a o}}{{P}_{a} {O}_{2}} \qquad ( 2 )$$

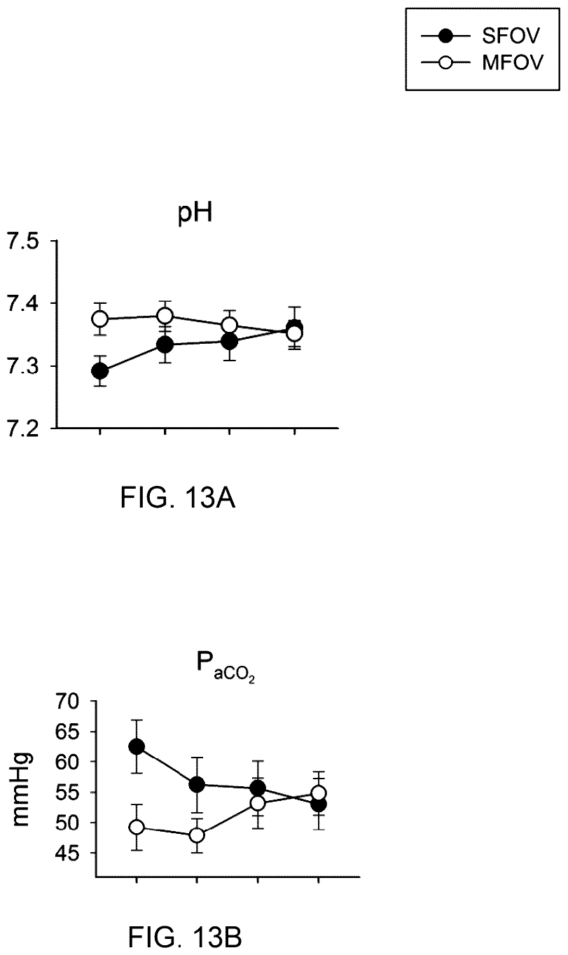

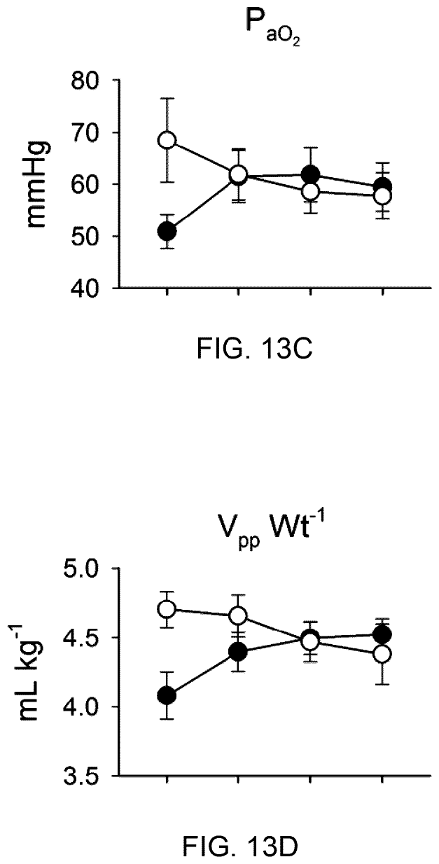

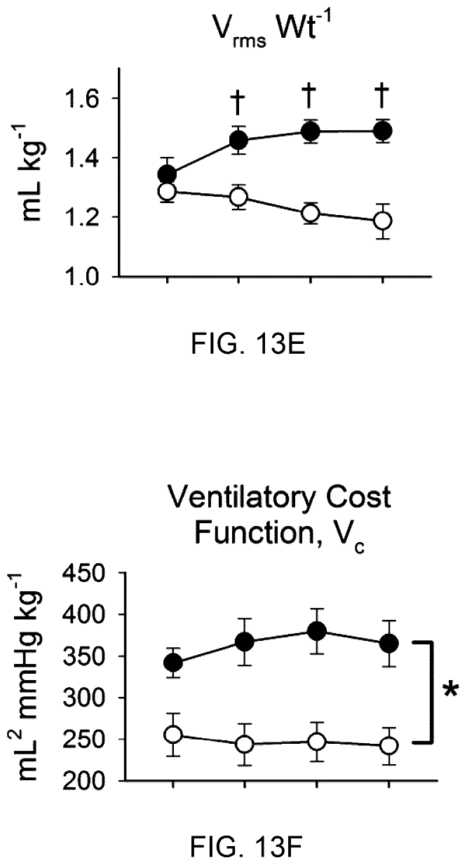

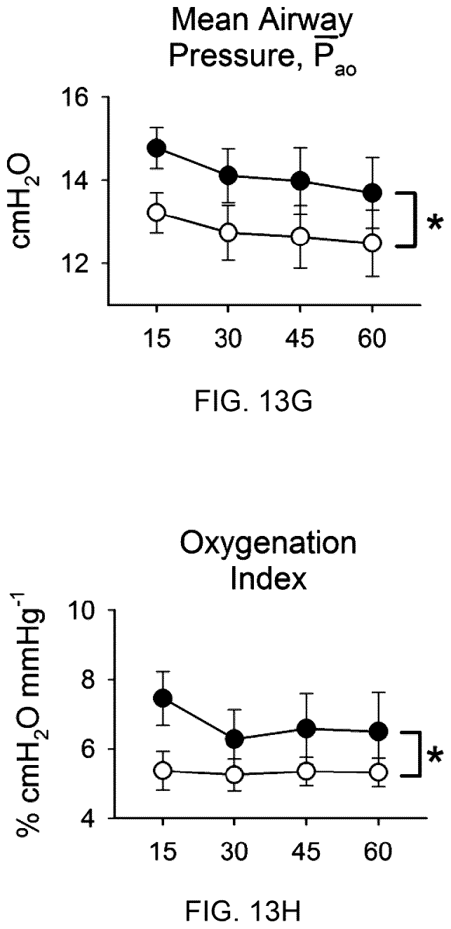

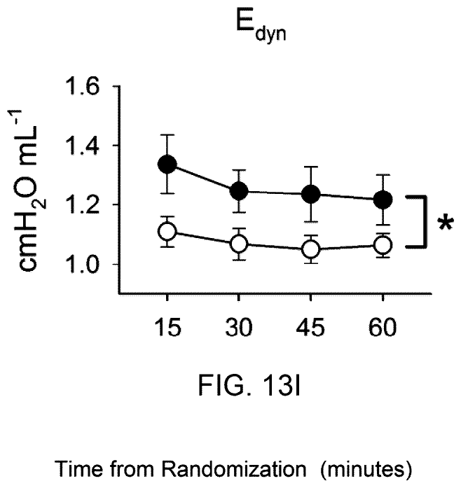

The Vᵣₘₛ was increased or decreased by 0.01 mL kg⁻¹ at 15 minute intervals, for every 1 mmHg above or below our target PₐCO₂ range of 45 to 55 mmHg. Since CO₂ elimination during HFOV is roughly proportional to V_{T}², we defined a ventilatory cost function (V_{C}) to compare the efficiency of gas exchange for SFOV and MFOV:

$${V}_{C} = ( {V}_{rms}^{2} {P}_{a} {CO}_{2} ) {Wt}^{- 1} \qquad ( 3 )$$

where Wt denotes body weight in kg. Thus for a given value of PₐCO₂, lower values of V_{C} indicate more efficient ventilation. The results are illustrated in FIGS. 13A-13I.

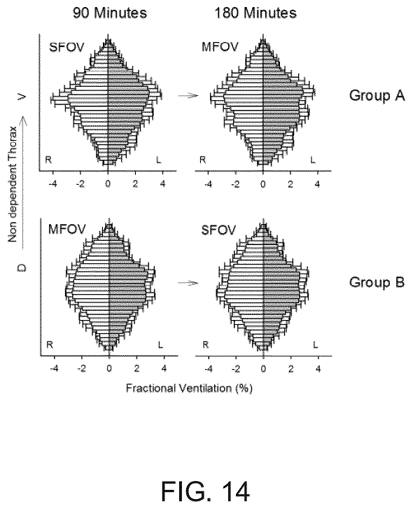

Electrical Impedance Tomographic (EIT) measurements (FIG. 14) were analyzed at the end of each 60 minute SFOV and MFOV period (i.e., at t=90 and 180 minutes of the protocol) for both crossover groups, using the GOE-MF II system with Thorascan software (CareFusion, Hochberg, Germany). The EIT data were acquired at a resolution of 32×32 pixels and a frame rate of 44 Hz. The EIT images were reconstructed using the GREIT image reconstruction algorithm with anatomic boundary shapes based on a transverse CT-scan of the chest of a lamb at the same height. Further details regarding this image reconstruction algorithm are present in Adler, et al., “GREIT, a unified approach to 2D linear EIT reconstruction of lung images,” Physiological Measurement 30 (2009) 535-555, the contents of which are hereby incorporated by reference herein. Thirty seconds of artifact-free representative data were chosen for analysis. To minimize cardiogenic disturbances, the EIT data was filtered using a 5th order digital Butterworth high-pass filter with 2 Hz cut-off frequency. From these filtered images, functional EIT (fLIT) images of ventilation were generated from the standard deviation of the time course of the impedance value within each pixel. From the fEIT images, the spatial distribution of ventilation within each of the 32 equally sized right-to-left and anterior-to-posterior slices could be determined. The geometric center of ventilation within each 32×32 fEIT image was computed using the technique of Frerichs et al. (Frerichs et al., “Monitoring perioperative changes in distribution of pulmonary ventilation by functional electrical impedance tomography”, Acta Anaesthesiol Scand 1998; 42: 721-726, the contents of which are hereby incorporated by reference herein).

Birth weights and umbilical cord blood gas values at delivery were compared between the two groups of lambs using two-tailed Student's t-tests. Group A were randomized to receive SFOV first followed by MFOV, while Group B first received MFOV followed by SFOV. At the end of the initial and washout 30 minute CMV periods, gas exchange and mechanics data for the two groups were compared using a two-way analysis of variance (ANOVA), with group (A vs. B) and period (initial vs. washout) as variable components. During the 60 minute SFOV and MFOV periods, gas exchange and mechanics data were analyzed via two-way repeated measures ANOVA, with treatment mode (SFOV vs. MFOV) and time (in 15 minute increments) as the variable components. EIT ventilation distributions were analyzed via three-way ANOVA, with treatment mode, group, and hemithorax (left vs. right) as the variable components. If significance was obtained with ANOVA, post hoc comparisons were performed using the Tukey HSD criterion. Unless otherwise specified, all data are expressed as mean±S.D., and P<0.05 was considered statistically significant.

Of the thirteen lambs, seven were randomized to receive SFOV first followed by MFOV (Group A), and six were randomized to receive MFOV first followed by SFOV (Group B). There were no significant differences in birth weights or umbilical cord pH, PaCO2, and PaO2 values between the two groups (Table 1).

TABLE 1
Birth weights and umbilical cord blood gas data for the thirteen
lambs at time of delivery, separated into Groups A and B.

| | Group A (n = 7) | Group B (n = 6) | Significance |
|---|---|---|---|
| Wt(kg) | 3.18 ± 0.41 | 3.11 ± 0.39 | 0.77 |
| pH | 7.33 ± 0.06 | 7.33 ± 0.20 | 0.39 |
| PₐCO₂ (mm Hg) | 57.0 ± 3.7 | 61.6 ± 6.2 | 0.15 |
| PₐO₂ (mm Hg) | 31.9 ± 2.6 | 27.7 ± 8.9 | 0.29 |

Data are expressed as mean ± S.D. Significance assessed by t-test with the exception of pH, which was assessed by Wilcoxon Rank Sum Test. Wt: weight; pH: negative base 10 logarithm of the hydrogen ion concentration; PₐCO₂: arterial partial pressure of carbon dioxide; PₐO₂: arterial partial pressure of oxygen.

FIGS. 13A-13I summarize the gas exchange and mechanics data at 15 minute intervals for the SFOV and MFOV modalities. No significant differences were observed in arterial pH at any time point between the two modalities, although the PaCO2 was significantly higher for SFOV at 15 minutes compared to MFOV. However, normalized Vpp was significantly lower for SFOV compared to MFOV for the first 15 minutes after randomization. Normalized Vrms was significantly higher for SFOV compared to MFOV for all time points after 15 minutes. Finally VC, mPao, O1, and Edyn were significantly lower at all time points during MFOV compared to SFOV.

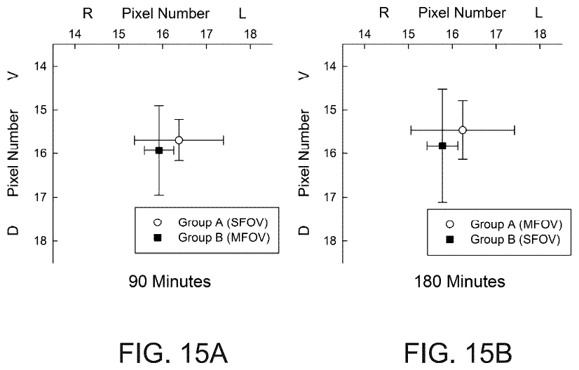

FIG. 14 shows the spatial distribution of ventilation at the end of each 60 minute SFOV or MFOV period (i.e., at 90 and 180 minutes of the protocol) for Groups A and B. Due to an electrode failure, one lamb from Group A was omitted from the EIT analysis. No significant differences were observed in ventilation distribution between the oscillatory modalities (SFOV vs. MFOV), groups (A vs. B), or right vs left and anterior vs posterior hemithoraces. There was also no significant difference in the geometric center of ventilation between the two groups (FIGS. 15A and 15B).

Premature lungs exhibit mechanical and spatial heterogeneity due to structural immaturity and surfactant deficiency. Conventional mechanical ventilation (CMV) in preterm infants may inadvertently worsen existing lung injury due to repeated alveolar overdistention and opening/closing of airways. By contrast, high frequency oscillatory ventilation (HFOV) achieves effective gas exchange with relatively high mean airway pressures to sustain lung recruitment, with small tidal volumes to prevent end-inspiratory overdistention. However, numerous clinical trials in adults and neonates with ARDS have shown that HFOV does not reduce mortality, despite physiological evidence of enhanced gas distribution, volume recruitment, and \dot{V}/\dot{Q} matching. Why such a theoretically promising mode of mechanical ventilation has so far failed to prove advantageous in clinical practice may be multifactorial: oscillation strategy, treatment end points, and/or operator skills are potential contributing factors. The failure of HFOV suggests suboptimal aeration and ventilation of the injured lung, potentially arising from variable regional effects of frequency, amplitude, and mean airway pressure in the setting of heterogeneous disease processes. There is no doubt that the mechanical complexity of the heterogeneous lung is fundamental to the distribution of ventilation.

As shown in FIG. 2, lung function and gas exchange would improve if volume oscillations are applied at multiple frequencies simultaneously, rather than at a single high frequency. Multiple frequencies improve oxygenation and \dot{V}/\dot{Q} matching by distributing ventilation more evenly to different regions, according to their respective local mechanical properties. Multi-frequency oscillatory ventilation (MFOV) using a hybrid ventilator-oscillator results in a more uniform distribution of ventilation to the alveoli compared to traditional SFOV. The mechanism for such improved functional outcomes is optimization of regional gas distribution, due to inclusion of frequencies that are more appropriate for the local mechanical properties of the heterogeneous parenchyma.

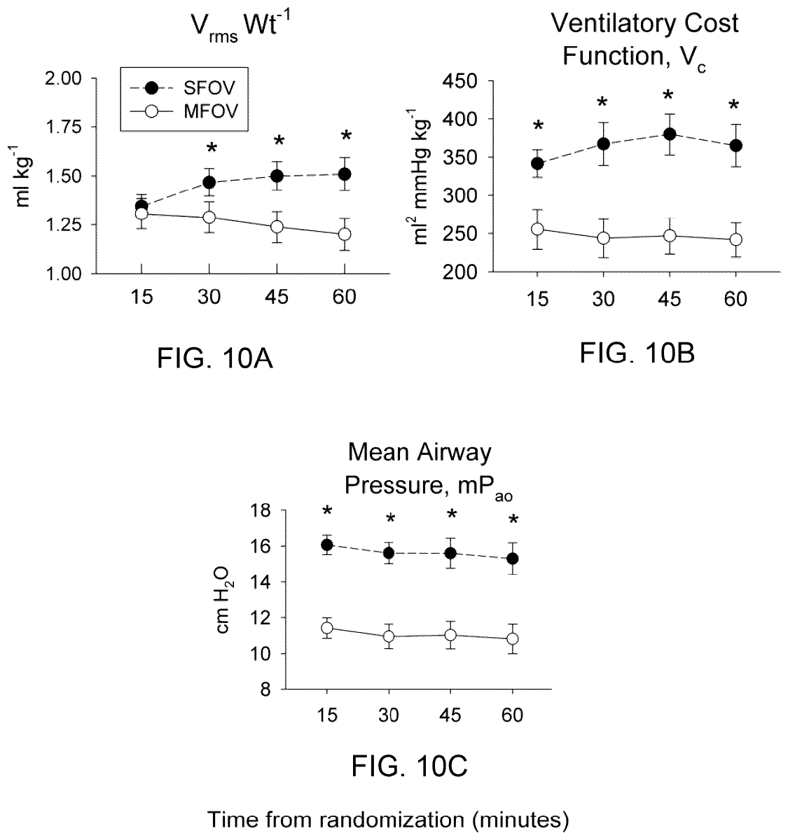

To assess the efficacy of MFOV, certain indices of gas exchange and mechanics may be evaluated. The ventilatory cost function (V_{C}) is defined as the product of ventilatory ‘power’ (Vᵣₘₛ²) multiplied by arterial CO₂ tension (PₐCO₂). This index is justified by empiric evidence that CO₂ elimination is roughly proportional to the square of tidal volume during HFOV. Our V_{C} index was significantly lower during MFOV compared to SFOV, consistent with more efficient CO₂ elimination (FIG. 10B). More efficient CO₂ elimination in MFOV was due largely to the increased Vᵣₘₛ required of the SFOV modality compared to MFOV in order to maintain PₐCO₂ within our desired target range of 45 to 55 mm Hg: there were no significant differences in the peak-to-peak volume excursions or PₐCO₂ between SFOV and MFOV after 15 minutes (FIG. 10A).

The OI and P̅ₐₒ were significantly lower at all time points during MFOV compared to SFOV, with no significant differences in PₐO₂. These findings indicate that MFOV facilitates more efficient oxygenation at lower distending pressures. The E_{dyn} was also significantly reduced during MFOV, although we cannot be certain whether lower E_{dyn} was the result of enhanced lung recruitment or reduced parenchymal strain-stiffening. The lower E_{dyn} further suggests that the enhanced spectral content of the MFOV waveforms may prevent time-dependent derecruitment of lung units, as the higher frequency components may act as a dithering mechanism to keep lung units opened.

The mechanism for improved gas exchange and global mechanics during MFOV provides improvement of regional gas distribution, due to the presence of additional frequencies that are more appropriate for the local mechanical properties of the heterogeneous parenchyma. The primary determinant of ventilation distribution in the injured lung is the distribution of regional mechanical properties of the airways and parenchyma, such as resistance, inertance, and elastance. Local ventilation distribution becomes highly frequency-dependent in the presence of regional mechanical heterogeneity. Thus, the most effective frequency for optimal gas exchange can vary from region to region. Oscillation at a single high frequency (i.e., standard HFOV) results in large portions of a heterogeneous lung being simultaneously underventilated and overventilated, with corresponding decrements in gas exchange and worsening injury. Previous methods using computational models indicate that small amplitude volume oscillation at a single, arbitrary frequency is not suitable for reaching the majority of the gas exchanging regions in spatially heterogeneous lung. Thus MFOV is better suited to complement the heterogeneous mechanics of the immature lung, by allowing the local impedances of the injured parenchyma to selectively filter out flows of ‘less-desirable’ frequencies. As a result, flows at frequencies more optimal for a particular region pass through and participate in gas exchange.

Despite the improved metrics of gas exchange and mechanics with MFOV, the ventilation distribution as assessed with EIT did not demonstrate significant differences between the SFOV and MFOV modalities. However, EIT provides a low-resolution assessment of ventilation distribution based on changes in electrical impedance, and only within a single cross-sectional slice of the thorax. Despite its utility as a potential bedside tool for rapid, noninvasive quantification of regional aeration, EIT may not describe ventilation distribution appropriately during HFOV modalities due to its 16-electrode array and maximum sampling rate of 44 Hz. Alternative methods for assessing the spatial distribution of ventilation, such dynamic volumetric computed tomography during Xenon washout, can be used for the high temporal variations associated with MFOV.

Certain commercially-available oscillators generate waveforms with higher harmonics above the fundamental frequency, although there is no evidence to suggest that these harmonic frequency artifacts influence clinical efficacy. This disclosure, on the other hand, is directed to the efficacy found from the use of multiple specifically tuned frequencies or broadband waveform patterns.

While these measurements are compelling, they are limited by: 1) a small sample size; 2) a MFOV waveform consisting of only three frequencies that were not optimized for the heterogeneous lung; and 3) minimal information on the mechanical properties of these preterm lungs. Despite these deficiencies, the data indicate that MFOV has distinct advantages as a ventilator modality in preterm lungs compared to traditional HFOV, and maintains lung recruitment at lower mean airway pressures. A larger sample size in an animal model more representative of heterogeneous clinical ARDS would further strengthen the concept of MFOV, and establish its use in eventual human clinical trials.

Mechanical heterogeneity in the injured lung has important implications for optimal treatment strategies in ARDS. This oscillatory modality improves gas exchange, specifically with regard to convective ventilation. With adjustments in oscillatory pressure amplitude and mean airway pressure, MFOV can improve gas exchange while minimizing the detrimental effects of cyclic alveolar overdistention and derecruitment.

MFOV provides in improved gas exchange at lower distending pressures in an animal model of preterm lung injury. MFOV has far-reaching implications for both pulmonary medicine and anesthesia. For example, MFOV is not limited to being a treatment solely for pediatric or adult ARDS, but is useful in the ventilator management of other diseases that affect the lungs in a heterogeneous manner such as asthma, COPD, or pneumonia. Moreover, MFOV can more efficiently penetrate ‘difficult-to-reach’ regions of the lung has implications for the optimal delivery of aerosols and drugs, such as beta agonists, steroids, or even inhaled volatile anesthetics.

MFOV is a more efficient ventilatory modality in preterm lungs compared to SFOV, and maintains lung recruitment at lower mean airway pressures. The spectral content of MFOV waveforms can be enhanced to improve gas exchange and result in less injurious ventilation compared to more conventional ventilation and oscillation strategies. MFOV can significantly change the care of critically-ill, ventilated patients.

While preferred embodiments and implementations of the invention have been set forth for purposes of illustration, the foregoing description should not be deemed a limitation of the invention disclosed herein. Accordingly, various modifications, adaptations and alternatives may occur to one skilled in the art without departing from the spirit and the scope of the present invention.

What is claimed is:

1. A system comprising:
an oscillatory ventilator configured for oscillating at plurality of specifically tuned frequencies simultaneously a ventilation gas for delivery to a pulmonary region of a patient; and
a ventilator control system, in communication with the oscillatory ventilator, to control a waveform input for the oscillatory ventilator, wherein the waveform input comprises the plurality of specifically tuned frequencies.

2. The system of claim 1, wherein the waveform further comprises of an amplitude and phase associated with each of the plurality of frequencies, wherein the amplitude and phase for each of the plurality of frequencies can be independently configured.

3. The system of claim 2, wherein one of the amplitude and phase associated with each of the plurality of frequencies is alterable in response to physical or physiologic changes in the patient.

4. The system of claim 1, wherein the ventilator control system further comprises, at least two signal generators wherein each signal generator produces a signal comprising of a specifically tuned frequency, amplitude, and phase that are combined to produce the waveform input.

5. The system of claim 1, wherein information about a status of a patient's respiratory system is used to determine the plurality of specifically tuned frequencies applied during a therapeutic treatment period.

6. The system of claim 1, wherein the ventilator control system provides a first plurality of specifically tuned frequencies and a second plurality of specifically tuned frequencies that are different than the first plurality of specifically tuned frequencies during a therapeutic period.

7. The system of claim 1, wherein the ventilator control system further comprises at least one method to determine the plurality of frequencies to be applied, and wherein the plurality of specifically tuned frequencies are determined and applied independently of each other, and wherein the plurality of specifically tuned frequencies are each fundamental frequencies.

8. The system of claim 1, and further comprising at least one sensor for measuring the ventilation gas being delivered to a pulmonary region of the patient and providing the measurement to the ventilator control system for use in producing the waveform.

9. The system of claim 8, wherein the ventilator control system further comprises an input device for receiving a measurement from the at least one sensor and for receiving at least one measurement corresponding to physiological characteristics of the patient, wherein the physiological characteristics include at least one chosen from \dot{V}/\dot{Q} airway pressure, airway flow, mechanical impedance, delivered concentration of gas species, ventilation-to-perfusion ratio, ventilation distribution, pH, arterial tension of carbon dioxide, or arterial tension of oxygen, a computer in communication with the input device for processing information from the input device, and output device in communication with the computer for controlling the oscillatory ventilator.

10. The system of claim 1, wherein the oscillatory ventilator further comprises a proportional solenoid valve for translating the waveform input from the ventilation control system to a ventilation gas waveform that corresponds to the waveform input.

11. A method comprising:
producing a waveform input comprising a plurality of specifically tuned characteristics; and
oscillating with an oscillatory ventilator gas for delivery to a pulmonary region of a patient based on the waveform input.

12. The method of claim 11, wherein the plurality of specifically tuned characteristics of the waveform comprise a frequency, an amplitude, and a phase.

13. The method of claim 11, wherein the waveform comprises of a plurality of specifically tuned frequencies with each of the plurality of specifically tuned frequencies further comprising an amplitude, phase, and power.

14. The method of claim 13, wherein the waveform further comprises a plurality of specifically tuned frequencies applied simultaneously to the oscillatory ventilator.

15. The method of claim 11, and further comprising treating the patient with one or more alternating periods of conventional ventilation, single frequency oscillatory ventilation, and multi-frequency oscillatory ventilation comprising oscillating with the oscillatory ventilator gas for delivery to the pulmonary region of the patient based on the waveform input.

16. The method of claim 11, and further comprising tuning a plurality of frequencies based on physiological characteristics of the patient during a therapeutic treatment period.

17. The method of claim 16, wherein the physiological characteristics include at least one chosen from \dot{V}/\dot{Q} airway pressure, airway flow, mechanical impedance, delivered concentration of gas species, ventilation-to-perfusion ratio, ventilation distribution, pH, arterial tension of carbon dioxide, and arterial tension of oxygen.

18. A method of treating a patient, the method comprising:
producing a waveform input comprising a plurality of specifically tuned characteristics;
oscillating with an oscillatory ventilator a gas for delivery to a pulmonary region of the patient based on the waveform input; and
evaluating a status of the patient.

19. The method of claim 18, and further comprising modify at least one of the plurality of specifically tuned characteristics of the waveform in order to modify a waveform of the ventilation gas for delivery to the patient.

20. The method of claim 19, wherein modifying the at least one of the plurality of specifically tuned characteristics of the waveform further comprises at least one chosen from a frequency, an amplitude, and a phase of the waveform.
